SEQUENCE LISTING (11 sequences)

SEQ ID NO: 1 - protein, 9 aa, Unknown
  functioning as an epitope binding with a specific antibody
YPYDVPDYA

SEQ ID NO: 2 - protein, 10 aa, Unknown
  functioning as an epitope binding with a specific antibody
EQKLISEEDL

SEQ ID NO: 3 - protein, 6 aa, Unknown
  functioning as an epitope binding with a specific antibody
HHHHHH

SEQ ID NO: 4 - protein, 11 aa, Unknown
  functioning as an epitope binding with a specific antibody
MASMTGGQQMG

SEQ ID NO: 5 - protein, 7 aa, Unknown
  functioning as an epitope binding with a specific antibody
DYDDDDK

SEQ ID NO: 6 - protein, 7 aa, Unknown
  functioning as an epitope binding with a specific antibody
HNHRHKH

SEQ ID NO: 7 - protein, 14 aa, Unknown
  functioning as an epitope binding with a specific antibody
GKPIPNPLLGLDST

SEQ ID NO: 8 - protein, 11 aa, Unknown
  functioning as an epitope binding with a specific antibody
YTDIEMNRLGK

SEQ ID NO: 9 - protein, 15 aa, Unknown
  functioning as an epitope binding with a specific antibody
KETAAAKFERQHMDS

SEQ ID NO: 10 - protein, 6 aa, Unknown
  functioning as an epitope binding with a specific antibody
EYMPME

SEQ ID NO: 11 - protein, 13 aa, Unknown
  functioning as an epitope binding with a specific antibody
SQPELAPEDPEDC

(19) United States
(12) Patent Application Publication
(10) Pub. No.: US 2009/0317334 A1
(43) Pub. Date: Dec. 24, 2009

(54) OPTICAL DETECTION METHOD OR IMAGING METHOD OF IMPLANTED CELLS

(75) Inventors: Naoaki OKAMOTO, Tokyo (JP); Chino SASAOKA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/481,948

(22) Filed: Jun. 10, 2009

(30) Foreign Application Priority Data
Jun. 10, 2008 (JP) 2008-152071

(51) Int. Cl. A61K 49/00

(52) U.S. Cl. 424/9.6; 424/9.1

(57) ABSTRACT

There is provided a method of an animal model experiment to observe cells implanted to an experimental small animal in which the position or spatial spread only of the implanted cells can be detected and observed with time progress and with sufficient accuracy while a plurality of kinds of the implanted cells can be distinguished from one another. The inventive method of in vivo detecting implanted cells implanted into the body of a small animal comprises steps of preparing cells used as the implanted cells into which a gene expressing a tag of a part of a peptide or a protein on a cell membrane is introduced; implanting the implanted cells to an arbitrary site of the small animal; and detecting the tags in the small animal. The tags may be detected by dosing the small animal with fluorescently labeled antibodies to the tags and imaging the fluorescence from the antibodies.

### TECHNICAL FIELD

The present invention relates to a method of in vivo detection of cells implanted into a small animal used as an experimental small animal, for example, a mouse, a rat, a guinea pig, and a rabbit, etc. (hereafter referred to as “implanted cells”), and more specifically, to a method for optical detection or imaging of implanted cells.

### BACKGROUND ART

In the fields of medical, pharmacological or biological researches, there are often carried out an animal model experiment in which arbitrary tumor cells or other cells are implanted to an experimental small animal and then the implanted cells are observed or tracked in the body (including the body surface, the same in the followings) of the experimental small animal. In this experiment, the implanted cells grow, metastasize, proliferate or become extinction under the environment in a living animal's body, and thus, through the observation of the behaviors (growth, metastasis, proliferation or extinction) of the implanted cells, one can obtain useful data concerning the in vivo mechanism of metastasis, growth, multiplication or extinction of cells (especially tumor cells). In particular, in the field of development of antineoplastic drugs, the size and position of a tumor of an animal into which tumor cells have been implanted are measured while an arbitrary drug (for example, a drug for a candidate of an antineoplastic drug) are dosed to the animal, and their measurement results are used for the evaluation of the effect of the arbitrary drug to the tumor cells.

In an animal model experiment to observe cells implanted into an experimental small animal as described above, conventionally, as a concrete way of observing the implanted cells, a method including the slaughter of an experimental small animal, the excision of a tumor and the measuring of the position, volume or mass of the cells has been employed (e.g., patent documents 1). However, recently, according to the progresses of various in vivo bio-imaging techniques and gene recombination techniques, non-invasive or low-invasive, temporal measurements of the position, volume or mass of implanted cells have been tried by means of the progressed techniques. For instance, non-patent document 1 reported that the imaging of tumor cells within a small animal was succeeded by dosing a small animal into which tumor cells had been implanted with a fluorescence reagent prepared to react with an enzyme (cathepsin D) produced and released by the tumor cells. Also, non-patent document 2 showed that, when leucocytes having been labeled with a fluorescent dye were dosed to a small animal, the tracking of the dosed leucocytes within the small animal was possible by the fluorescence tomography, etc. (The flocking of the fluorescently labeled leucocytes to a tumor was shown by fluorescence imaging.) Further, it has been proposed to employ the imaging with radioactive substances, such as the radioactive labeling, PET, as the technique for the detection of tumor cells in a body of a small animal (for example, see non patent documents 3 and 4). Moreover, in patent documents 2-5, it has been proposed to implant, into an experimental small animal, tumor cells prepared to produce therein the fluorescent proteins, such as GFP, or the luminescent proteins, such as luciferase, through gene introduction technique, and to observe the behaviors of the implanted tumor cells by the imaging of the fluorescence or luminescence from the fluorescent proteins or luminescent proteins expressed within the implanted tumor cells.

Especially, in the fluorescence or luminescence imaging method using the above-mentioned fluorescent proteins or luminescent proteins, since only the implanted cells “glow” and the cells after repetitive cell division and multiplication in the body of an animal continue glowing similarly to the cells at the implantation, the behavior of an implanted cell can be tracked and observed with time progress and with sufficient accuracy (In the imaging method using a fluorescent substance which reacts specifically with an enzyme produced in implanted tumor cells or fluorescently labeled leucocytes, the light is emitted also from sites other than the implanted cells, and therefore, it is not always possible to image only implanted cells, so that the accuracy will lower). Furthermore, the method using a fluorescent protein or a luminescent protein is easy and advantageous in the preparation and operation of an experiment because neither the handling of radioactive substances nor large-scale apparatus and facility for detecting radiation is required.

### PRIOR ART DOCUMENTS

Patent documents 1: Japanese patent laid-open publication no. 2003-261448
Patent documents 2: Japanese patent No. 3786903
Patent documents 3: Japanese patent laid-open publication no. 2001-517090
Patent documents 4: Japanese patent laid-open publication no. 2002-534398
Patent documents 5: Japanese patent laid-open publication no. 2005-6660
Non-patent document 1: Tung, Ching-Hsuan, and other three persons, Cancer Research 60, 4953-4958, Sep. 1, 2000.
Non-patent document 2: Swirski, Filip-K, and other six persons, PLos ONE 10, e1075, October,
Non-patent document 3: Saibo Kogaku Bessatsu, Jikken Protocol Series “Counterattack of RI”, Editorial supervision: Seiji Okada, Shujunsha, p. 132-137, December, 2007
Non-patent document 4: Saibo Kogaku Bessatsu, Jikken Protocol Series “Counterattack of RI”, Editorial supervision: Seiji Okada, Shujunsha, p. 138-147, December, 2007

### SUMMARY OF INVENTION

By the way, in an animal model experiment to observe cells implanted to an experimental small animal as described above, various cells may be used as the implanted cells. For example, in the investigation of influences of a certain drug on a plurality of kinds or lines of tumor cells for evaluating a usefulness of the drug as an antineoplastic drug, tumor cells of the respective kinds to be investigated are implanted to small animals, and then their behaviors will be observed.

In the non-invasive or low invasive imaging methods of observing cells implanted to an experimental small animal known so far, however, it is difficult to distinguish a plurality of kinds of implanted cells in the simultaneous imaging of those cells in one individual animal. For instance, in the above-mentioned imaging with radioactive substances, such as the radioactive labeling and PET, it is not possible to identify a plurality of kinds of implanted tumor cells simultaneously at the detection thereof. Further, in the case of a method of detecting an implanted cell in a fluorescence or luminescence imaging experiment using implanted cells producing a fluorescent protein or a luminescent protein, the kinds of fluorescent proteins or luminescent proteins expressible in the implanted cell are limited so that the number of the options of wavelengths of the detectable light is comparatively small, and thus, it is not always possible to establish an experimental condition enabling the identification of a plurality of kinds of implanted cells. Especially, in the in vivo imaging, it is required to choose, as the detection light, the light of a wavelength highly permeable through a living organism, and therefore, if proteins emitting the light of a wavelength highly permeable to a living organism can not be produced as fluorescent proteins or luminescent proteins within an implanted cell, no accurate or clear image of implanted cells will be obtained. Actually, in the experiments with implanted cells producing fluorescent proteins or luminescent proteins reported so far, the number of the kinds of implanted cells detectable in one time is up to one, and thus, in order to carry out the above-mentioned experiment for a plurality of kinds of cells, it is required to prepare one animal individual for each of the kinds of cells.

Thus, in an animal model experiment to observe cells implanted to an experimental small animal as described above, if there is a method enabling the detection and/or observation of a position or a spatial spread of only implanted cells with time progress and with sufficient accuracy and also the identification of a plurality of kinds of the implanted cells, it is expected that one can carry out such an animal model experiment to observe implanted cells in an experimental small animal in various manners while using more various kinds of cells than ever. According to the present invention, in order to make it possible to identify a plurality of kinds of implanted cells simultaneously in an animal model experiment to observe cells implanted to an experimental small animal, a cell into which a gene expressing a peptide or a protein to serve as a tag on its cell membrane has been introduced is employed as an implanted cell. Thereby, there is provided a novel method of detecting the positions and spatial spread of implanted cells by detecting tags having been made “grow” on the cell membranes. For peptides or proteins to be used as the tag, various kinds can be selected, and therefore, it become possible to track and observe the positions and spatial spread of the implanted cells of the respective kinds in the body of a small animal with time progress, for instance, by making different tags grow on different kinds of cells to be implanted; implanting the plurality of kinds of cells to one animal individual; and detecting the tags on the cell membranes.

In accordance with one aspect of the above-mentioned present invention, there is provided a method of in vivo detecting cells implanted into the body of a small animal, characterized by comprising steps of preparing cells, to be used as the implanted cells, into which a gene expressing a tag of a part of a peptide or a protein on a cell membrane has been introduced; implanting the implanted cells to an arbitrary site of the small animal; and detecting the tag in the small animal. In this structure, the small animal may be an arbitrary animal for an experiment usually used in this field, and the implanted cells may be tumor cells of a mammalian origin, such as a human, a mouse and a rat. The tag to be expressed on the cell membrane of the implanted cells may be a protein with antigenicity, such as avidin, maltose binding protein and glutathione S transferase (GST), or a peptide arbitrarily chosen from the group consisting of HA-tag peptide, c-Myc-tag peptide, His-tag peptide, T7-tag peptide, Flag-tag peptide, metal affinity-tag peptide, V5-tag peptide, VSV-G-tag peptide, S-tag peptide, Glu-Glu-tag peptide and HSV-tag peptide (For the amino acid sequences of the peptides, refer to the column of Mode for carrying out the invention).

In the above-mentioned aspect of the present invention, although the basic processes are the same as those of a conventional animal model experiment to observe cells implanted to an experimental small animal, what should be focused are that a peptide or a protein as listed above is made expressed on a cell membrane as a tag, and that the tag grown on the cell membrane is detected as a mark of an implanted cell. As already noted, in this field, it has been already possible to express a plurality of kinds of proteins and peptides selectively on a cell membrane of an arbitrary cell by the gene introducing technique. Thus, by using arbitrary ones selected from various kinds of the proteins and peptides as described above for the mark of an implanted cell, instead of a fluorescent protein or luciferase which has only a narrow range of selectivity, it becomes possible to characterize a variety of kinds of cells individually. Further, since the “tags” of proteins and/or peptides will be maintained in each cell even after the division and proliferation of the implanted cells in a living body, the implanted cells will be detected by detecting the “tags” in an arbitrary manner, and thereby, the tracking and observing of the behaviors of the implanted cells sequentially with time progress in one animal individual become possible.

Accordingly, it should be understood that the above-mentioned inventive method may be designed to perform preparing a plurality of kinds of cells as the implanted cells such that each of the kinds will express a different tag from the others; implanting the kinds of cells into a single small animal individual; and detecting the mutually different tags individually in the small animal individual, and thereby, it becomes possible to implant cells of a plurality of kinds into a single small animal individual and to simultaneously track and observe the behaviors of the respective kinds of cells. However, of course, even when only one kind of cells is implanted at one time, a cell expressing a tag on a cell membrane may be used as an implanted cell. Also, even in a case that one kind of cells is implanted, a plurality of cell groups prepared to express mutually different tags may be employed as implanted cells (For example, an experimental manner of implanting cells to a plurality of sites in one animal individual can be considered). In the above-mentioned inventive structure, in the step of detecting the tags in a small animal, typically, the “tags” may be optically detected from the outside of the small animal, for example, by means of a stereoscopic microscope or an optical microscope. Since the optical detection does not require a shielded room and a large-scale experimental device for the detection method using a radioactive substance, an experiment can be performed more simply and safely than the detection method using a radioactive substance. For the optical detection of the tags, while an arbitrary method may be employed, typically, an image of a small animal is acquired and a region occupied by the tags is detected in the image, and thereby, it becomes possible to easily determine the position or spatial spread of the implanted cells in the small animal.

Moreover, in the step of detecting the tags in a small animal according to the present invention, while an arbitrary method known in this field may be used as the way of detecting the tags, in one preferable mode, the tags may be detected by dosing the small animal with antibodies to the respective tags and detecting the antibodies having bound to the respective tags in the small animal. As well known in one skilled in the art, an antibody binds specifically to an antigen, and thus, in accordance with the above-mentioned operation, the antibodies to the respective tags will automatically accumulate on the implanted cells in the small animal. Thus, by detecting an antibody, it becomes possible to “specifically” detect implanted cells or a group of implanted cells with sufficient accuracy.

Furthermore, the way of detecting the above-mentioned antibodies may be achieved by providing the antibodies with a fluorescent label and detecting the fluorescence from the antibodies having bound to the tags in the small animal. According to this manner, it becomes possible to easily determine the accumulation region of the antibodies, i.e., the region occupied by the implanted cells by means of an arbitrary fluorescence imaging technique, etc. What should be understood is that, because of the use of the fluorescently labeled antibodies whose fluorescent label can be selected from a wide range of fluorescent substances, one can achieve the highly sensitive and accurate observation of implanted cells with a fluorescent substance having a fluorescence wavelength which is highly permeable through the living organism, while preventing influences of a nonspecific reaction and an autofluorescence.

In this regard, it should be understood that, in the way of detecting tags, the tags may be detected simply by labeling the tags fluorescently and detecting the fluorescence from the tags. For example, when fluorescently labeled molecules which specifically bind to the tags, not antibodies, are dosed to a small animal, the fluorescently labeled molecules will accumulate on implanted cells (For example, when avidin is used as a tag, fluorescently labeled biotin may be used). Then, by detecting the fluorescence by means of an arbitrary fluorescence imaging technique, the presence, position and spatial spread of the implanted cells will be detected.

And, in detecting tags by the above-mentioned fluorescence imaging technique, typically, the tags may be detected by acquiring a fluorescence image of a small animal through the fluorescence observation of the small animal and detecting a region occupied by the fluorescence from the tags in the fluorescence image. According to this manner, the substantial fluorescence (fluorescence except an autofluorescence, etc.) is considered to come from the implanted cells, so that one can accurately and easily conduct the tracking and observing of the behaviors of the implanted cells with time progress while identifying various kinds of implanted cells.

In this regard, as shown in the experimental example explained later, it has been confirmed that, in the inventive method, through detecting a region occupied by the fluorescence from tags using a fluorescence imaging technique, the positions and size of implanted cells in a small animal can be estimated based on the region occupied by the fluorescence. Thus, in one aspect of the present invention, there is provided a method characterized by measuring the position or spatial spread of implanted cells based on a region occupied by detected tags. According to this method, it becomes possible to measure the position or spatial spread of implanted cells without actually extracting the implanted cells, and thereby the efficiency of experiments (regarding the reduction of the number of animal individuals to be used, etc.) becomes improved, and also, it becomes advantageously available to continue a sequential measurement of the position and spatial spread of implanted cells in one animal individual.

In one embodiment for realizing the effect of a series of above-mentioned manners according to the present invention, it is preferable that, in a method of in vivo detecting cells implanted into a body of a small animal, there may be sequentially performed steps of: preparing cells to be used as the implanted cells into which a gene expressing a tag of a part of a peptide or a protein on a cell membrane has been introduced; implanting the implanted cells into an arbitrary site of the small animal; dosing the small animal with fluorescently labeled antibodies to the tags; and acquiring a fluorescence image of the small animal and detecting a region occupied by the implanted cells by detecting a region occupied by fluorescence from the antibodies having bound to the tags in the fluorescence image. Further, in this embodiment, there may be carried out preparing a plurality of kinds of cells expressing mutually different tags as the implanted cells; implanting those kinds of cells to a single small animal individual; labeling antibodies to the respective mutually different tags with the fluorescent substances having mutually different fluorescence wavelengths; and detecting regions occupied by fluorescence from the respective antibodies having the mutually different fluorescence wavelengths and having bound to the mutually different tags in the small animal individual, so that the regions occupied by the implanted cells can be detected for the respective kinds of the cells. In the above-mentioned embodiment, the site in the body of the small animal to which the cells are to be implanted may be an arbitrary site, such as a subcutaneous part, a head, a neck, a leg, a tail, internal organs, a body cavity, etc. of an animal.

In general, according to the above-mentioned inventive method, through employing a cell in which a tag is made grow on its cell membrane as an implanted cell, various experimental manners can be considered than ever. In either of conventional animal model experiments to non-invasively or low invasively observe cells implanted to an experimental small animal, it was very difficult to distinguish the kinds of cells at the in vivo detection of the implanted cells. According to the above-mentioned features of the present invention, however, the simultaneous use of a plurality of kinds of tags enables the detection of the implanted cells with the cells of a plurality of kinds being distinguished at one time, and thereby, an animal model experiment to observe cells implanted to an experimental small animal using various combinations of implanted cells becomes performable. In that case, in particular, through the use of an optical detection method or a fluorescence imaging technique, it becomes possible to make experimental procedures and facilities simple, so that the environment in which the above-mentioned animal model experiments can be performed is expanded, and consequently, it is expected that an animal model experiment of such type will be performable in much wider area, and then, it is thought that the present invention will contribute also to the technical promotion in the medical fields, such as the promotion of development of new drugs.

Other objects and advantages of the present invention will become apparent from the following explanations of the preferable embodiments of the present invention.

### BRIEF EXPLANATIONS OF THE DRAWINGS

FIG. 1A-FIG. 1D are bright field images and fluorescence images of model mice in which HT1080 cells, prepared such that HA-tag peptides were made expressed on the cell membranes, were implanted into the subcutaneous parts by injection, and then anti HA-tag peptide antibodies fluorescently labeled with Alexa 750 were dosed. FIG. 1A and FIG. 1B are a bright field image and a fluorescence image of a mouse 3 days after the implantation of the cells, respectively, and FIG. 1C and FIG. 1D are a bright field image and a fluorescence image of a mouse 14 days after the implantation of the cells, respectively. The fluorescence images were captured with Fluorescence Bio-observation system OV110 using an attached filter for 750 nm.

FIG. 2 shows a relation between the areas of fluorescing regions and the weights of tumors which existed in the corresponding fluorescing regions (measured after extracted out) in the fluorescence images of model mice in which HT1080 cells, prepared such that HA-tag peptides were made expressed on the cell membranes, were implanted into the subcutaneous parts by injection, and then anti HA-tag peptide antibodies fluorescently labeled with Alexa 750 were dosed. The ordinate indicates an arbitrary unit and the abscissa indicates gram. In the computation of the area of a fluorescing region, the ROI analysis in the software attached with the fluorescence bio-observation system OV110 was used, in which the “area of a fluorescing region” is defined as the area of a region occupied by a tumor (a region exhibiting locally strong fluorescence intensity) encircled on a fluorescence image such that the average brightness value therein is rendered to fall in the range of 100-120. In the drawing, the solid line is the correlation line obtained by the least-squares method. The correlation coefficient was R²=0.925. In the shown example, the process for the subtraction of background light, etc. was not executed at the computation of the area of a fluorescing region because of the use of fluorescent dye whose background was low, but, if required, the process for the subtraction of background light, etc. may be executed.

FIG. 3A-FIG. 3B are fluorescence images of a model mouse in which HT1080 cells, prepared such that HA-tag peptides were made expressed on the cell membranes, and Hepa1-6 cells, prepared such that both HA-tag peptides and c-Myc-tag peptides were made expressed on the cell membranes, were implanted into the subcutaneous parts by injection, respectively, and then anti HA-tag peptide antibodies fluorescently labeled with Alexa 750 and anti c-Myc-tag peptide antibodies fluorescently labeled with Alexa 680 were dosed. FIG. 3A is a fluorescence image of only the fluorescence wavelength band of Alexa750, and FIG. 3B is a fluorescence image of only the fluorescence wavelength band of Alexa 680. In FIG. 3A and FIG. 3B, Region α is the same region, where it is considered that a tumor of Hepa1-6 cell has been formed, and in FIG. 3A, it is considered that a tumor of HT1080 cell has been formed in Region β.

### MODE FOR CARRYING OUT THE INVENTION

In the followings, the present invention will be explained in detail with respect to some preferable embodiments with reference to the attached drawings. The same references show the same sites in the drawings.

### Outline of the Animal Model Experiment

In general, in the animal model experiment to observe cells implanted to an experimental small animal of this embodiment, (a) the step of preparation of implanted cells; (b) the step of implantation of the implanted cells to an experimental small animal; and (c) the step of observation of the implanted cells are sequentially carried out similarly to a conventional similar experiment. Typically, the implanted cells are arbitrary tumor cells, and in the observation step of the implanted cells, how the cells implanted in the small animal grow, metastasize, proliferate or become extinct is observed with time progress. However, especially in the experiment according to the present invention, cells into which a gene expressing a tag of a part of a peptide or a protein on the cell membrane has been introduced are employed as the implanted cell, and the identification or detection of the implanted cells in the body of a small animal is performed by detecting the tags on the cell membranes. Since it is expected that the tags are expressed in substantially all descendent cells of the implanted cells (cells after division/proliferation), it is possible in the experiment of this embodiment to observe the implanted cells or the groups of the implanted cells continuously and at a constant sensitivity even after growth, metastasis and proliferation of the cells (For instance, in a case that a fluorescent label is simply given to an implanted cell, its descendent cells would not carry the fluorescent label or the amount of the label per cell reduces gradually, leading to the reduction of the detection sensitivity).

Further, in the experiment of this embodiment, the detection of the tags in the body of the small animal is carried out by dosing the small animal with fluorescently labeled antibodies which have been prepared such that the tag is made their epitope; and observing the small animal by fluorescence imaging. The above-mentioned antibodies, after dosed to the small animal, are expected to bind specifically to the tags on the cell membranes of the implanted cells, thereby accumulating on the peripheries of the implanted cells. Then, through observing the small animal under this condition by fluorescence imaging, the positions and spatial spread of the implanted cells will be revealed from the distribution of the fluorescence emitted from the antibodies on the fluorescence image of the small animal.

In this respect, one should understand that, as described in the column of “Summary of the invention”, various substances are selectable as the tag which can be made grow on a cell membrane and also various fluorescent substances are selectable for the label which can be attached on an antibody to bind to the tag, and thus, by preparing a plurality of cells expressing mutually different tags as the implanted cells and labeling fluorescent substances having mutually different wavelength bands onto antibodies to the respective tags, an observation to detect a plurality of cells implanted simultaneously into one animal individual becomes possible with the plurality of the implanted cells being distinguished.

Hereafter, the concrete examples of the steps of the experiment are explained sequentially.

### Preparation of Implanted Cells

The implanted cells in the experiment may be prepared by the following processes.

(1) A gene which makes either of a protein or a peptide having an antigenicity and being able to function as a tag on a cell membrane expressed on the cell membrane is introduced into a plasmid vector which can make a protein or a peptide expressed on the cell membrane surface of a cell originating from a mammal, such as a human, a mouse and a rat (a plasmid vector designed so as to express a protein in which a region passing through a cell membrane and a region fixed on the outside of the cell membrane are united to each other). Such a protein may be e.g. avidin, maltose joint protein, glutathione S transferase (GST), and such a peptide may be (in the parenthesis, the amino acid sequence of each peptide is shown):

HA-tag peptide
(Tyr-Pro-Tyr-Asp-Val-Pro-Asp-Tyr-Ala),
C-Myc-tag peptide
(Glu-Gln-Lys-Leu-Ile-Ser-Glu-Glu-Asp-Leu),
His-tag peptide
(His-His-His-His-His-His),
T7-tag peptide
(Met-Ala-Ser-Met-Thr-Gly-Gly-Gln-Gln-Met-Gly),
Hag-tag peptide
(Asp-Tyr-Asp-Asp-Asp-Asp-Lys),
Metal affinity-tag peptide
(His-Asn-His-Arg-His-Lys-His),
VS-tag peptide
(Gly-Lys-Pro-Ile-Pro-Asn-Pro-Leu-Leu-Gly-Leu-Asp-
Ser-Thr),
VSV-G-tag peptide
(Thy-Thr-Asp-Ile-Glu-Met-Asn-Arg-Leu-Gly-Lys),
S-tag peptide
(Lys-Glu-Thr-Ala-Ala-Ala-Lys-Phe-Glu-Arg-Gln-His-
Met-Asp-Ser),
Glu-Glu-tag peptide
(Glu-Tyr-Met-Pro-Met-Glu),
HSV-tag peptide
(Ser-Gln-Pro-Glu-Leu-Ala-Pro-Glu-Asp-Pro-Glu-Asp-
Cys),

but not limited thereto. The way of introducing the gene into a plasmid vector may be performed by an arbitrary method which can be used in this field. In this connection, a plurality of kinds of tag expressing genes may be introduced into one plasmid vector. Further, when one wishes to identify and detect a plurality of cells or cell groups in one animal individual, a plurality of kinds of plasmid vectors are prepared such that a gene expressing a protein or a peptide to become a mutually different tag has been introduced into each of the vectors.
(2) The plasmid vector prepared in the process (1) is introduced into tumor cells originating from a mammal, such as a human, a mouse and a rat (Hela, HT1080, LLC, Hepa 1-6, etc.). The method for the introduction may be an arbitrary method available in this field, such as a method using a transfection reagent (Lipofectamine™, etc.), the electroporation method, etc. In this respect, when one wishes to identify and detect a plurality of cells or cell groups on one animal individual, plasmid vectors expressing different tags may be introduced into a plurality of cells or cell groups to be identified. Then, the cells into which the plasmid vectors have been introduced are cultivated and selected in a selective medium such as geneticin added medium.

### Implantation of Implanted Cells to an Mental Small Animal

About 10⁵-10⁷ of cells are collected from each of the medium in which the cell groups prepared so as to express arbitrary tags on a cell membrane have been cultivated as described above, and then implanted to an experimental small animal by hypodermic injection or intravenous injection. The small animal may be an arbitrary animal usually used in this field, for example, a mammal, such as a mouse, a rat and a rabbit. The animal to which the cells have been implanted is fed in a usual manner until the observation of the implanted cells.

### Preparation of Fluorescently Labeled Antibodies

As already noted, the detection of the implanted cells in the body of the animal into which the cells have been implanted is performed by dosing the animal with fluorescently labeled antibodies which specifically bind to the tags expressed on the cell membrane, and detecting the fluorescence from the antibodies in the animal. Such antibodies may be prepared as follows:

(1) Antibodies to proteins or peptides used as tags expressed on cell membranes (anti-avidin antibody, anti-GST antibody, anti-maltose binding protein antibody, anti-HA-tag peptide antibody, anti-c-Myc-tag peptide antibody, anti-His-tag peptide antibody, anti-T7-tag peptide antibody, anti-Flag-tag peptide antibody, anti-metal affinity tag peptide antibody, anti-V5-tag peptide antibody, anti-VSV-G-tag antibody, anti-S-tag peptide antibody, anti-Glu-Glu-tag peptide antibody, anti-HSV-tag peptide antibody, etch) are prepared by an arbitrary method (those may be purchased commercially), and to those antibodies, fluorescent dyes, such as Alexa series fluorescent dye, Cy series fluorescent dye, ATTO series fluorescent dye and rhodamine series fluorescent dye, are attached by an arbitrary method. In this regard, a fluorescent dye may be arbitrary, but, it is selected from those having a long fluorescence wavelength band, highly permeable through a living organism (650 nm or more), for performing in vivo fluorescence imaging. Further, in a case that a plurality of kinds of cell groups expressing proteins or peptides to be mutually different tags are implanted to one animal individual in order to identify and detect a plurality of cells or cell groups in the animal, dyes having mutually different fluorescence wavelength bands are attached to the respective antibodies to the different tags. For instance, a combination of the fluorescent dyes having mutually different fluorescence wavelength bands from one another may be Alexa680 and Alexa750; Cy5.5 and Cy7; or Vivotag680 and Vivotag750, but not limited thereto, and also, a combination of three or more kinds of fluorescent dyes may be employed.
(2) The fluorescently labeled antibodies are purified with a gel filtration column, such as BioGelP-30, or a ultrafiltration spin column, such as Microcon (Millipore Corp.), etc. in order to remove unreacted dyes.

### Detection of Implanted Cells—Fluorescence Observation of an Experimental Small Animal

The detection of the cells implanted in the animal's body is performed through the fluorescence observation of the animal dosed with the above-mentioned fluorescently labeled antibodies by fluorescence imaging technique wherein the fluorescence from the antibodies is imaged. Its procedures may be as follows:

(1) To an animal individual to which the implanted cells have been implanted, the fluorescently labeled antibodies to the tags expressed on the implanted cells are dosed by intravenous injection, intraperitoneal injection, etc.
(2) After the lapse of time for the antibodies to accumulate on the implanted cells in the animal body, the fluorescence observation of the small animal dosed with the fluorescently labeled antibodies is performed using an arbitrary fluorescence imaging device, equipped with a stereoscopic microscope or an optical microscope, e.g. OV110 (Olympus). Typically, a fluorescence image of the small animal in the fluorescence wavelength band emitted from the fluorescently labeled antibodies is captured, and stored in an arbitrary storage device for an image analysis to be carried out later. In this regard, in order to distinguish a plurality of cells or cell groups in one animal individual in their detection, a fluorescence image of the small animal is captured for each of the fluorescence wavelength bands of the fluorescently labeled antibodies corresponding to the tags expressed in the cells implanted into the animal, and then stored into a storage device. Further, when an image capturing device capable of color imaging is used for acquisition of images, a fluorescence image covering a plurality of fluorescence wavelength bands may be acquired at one time.
(3) In a captured fluorescence image, it is expected that a fluorescence image of the antibodies having accumulated on the peripheries of the implanted cells will be captured. Thus, the positions, sizes or distribution of the implanted cells may be measured from the data in the image with arbitrary image analysis software.

What should be understood in the above-mentioned experiment is that, as already mentioned, the tags are made expressed hereditarily on the cell membrane of each implanted cell as a marker of the implanted cell. According to this structure, since the fluorescence of the fluorescently labeled antibodies having been made react with the tags can be detected, it becomes possible to identify and detect each of kinds of the implanted cells having been implanted into one animal by selecting an arbitrary combination of a tag and a fluorescent dye labeled on the antibody from the wide range of the tags and the wide range of the fluorescent dyes. There, because it is expected that the antibodies substantially bind only to the tags present only on the implanted cells and because a fluorescent dye having the fluorescence wavelength band highly permeable through a living organism can be selected for the dye to be attached to the antibodies, it is also possible to make the detection accuracy of the implanted cells higher than the prior art. Further, because of the use of the fluorescence imaging in the detection of the implanted cells, the inventive experiment can be simply performed without requiring a large experimental facility or radioactive substances.

In order to verify the validity of the present invention explained above, the following experiments were carried out. In this regard, it should be understood that the following embodiments are only for illustrating the validity of the present invention, and not intended to limit the scope of the present invention.

### EMBODIMENTS

In the present embodiments, in the case where HT1080 cells were implanted to one model mouse, and in the case where HT1080 cells and Hepa1-6 cells were simultaneously implanted to one model mouse, the formations of tumors of the respective cells were observed in the respective model mice with the kinds of the cells being distinguished from one another. The operational processes were carried out as follows:

1. Plasmid vector (a) which can express HA (hem agglutinin antigen)-tag peptide on a mammalian cell membrane surface, and plasmid vector (b) which can express both HA-tag peptide and c-Myc-tag peptide on a mammalian cell membrane surface were prepared by modifying a plasmid vector, pDisplay vector (Invitrogen Co.), designed to express a protein which has a region passing through a cell membrane and a region fixed on a cell membrane.
2. The plasmid vector (a) and the plasmid vector (b) were introduced into HT1080 cells and Hepa1-6 cells, respectively, with a reagent for transfection, lipofectamine 2000 (invitrogen), in accordance with its instruction manual.
3S. The HT1080 cells and Hepa6 cells into which the above-mentioned plasmid vectors were introduced each were cultivated with MEM culture medium (+10% FBS, +penicillin streptomycin) containing 600 □g/ml of geneticin (G-418, Invitrogen Co.), and the cells incorporating the plasmid vectors were selected with drugs.
4. The above-mentioned HT1080 cells and Hepa1-6 cells were subcultured under the condition of 5% CO₂ at 37° C. using the same culture medium. After this, for the respective cell kinds, 2×10⁶ of cells (100 □L) were collected and implanted into a back subcutaneous part or an overarm portion subcutaneous part of a bacb/c line nude mouse by injection.
5. On the other hand, for fluorescently labeled antibodies to be dosed to the above-mentioned mice, there were prepared commercially available anti-HA-tag peptide antibodies (COVANC, HA.11 Monoclonal Antibody), which was made react with Alexa Fluor (registered trademark) 750, succinyrnidil ester reactive dye (Invitrogen Co.) in accordance with its instruction manual, and commercially available anti-c-Myc-tag peptide antibodies (MBL, Anti-Myc-Tag), which was made react with Alexa Fluor (registered trademark) 680, succinymidil ester reactive dye (Invitrogen Co.) in accordance with its instruction manual. These fluorescently labeled antibodies each were gel-filtered through a column of 10 mm×300 mm filled with BioGel P-30 gel (Biorad) swelled with PBS for the removing of unreacted fluorescent dyes, and the fluorescently labeled antibodies' elution sites were separately collected.
6. To the model mouse in which a tumor was formed through the implantation of the HT1080 cells to the subcutaneous part by injection, the anti-HA-tag peptide antibodies fluorescently labeled with Alexa 750 were intravenously dosed at 15 Hg/animal to react with the HT1080 cell tumor which expressed HA-tag peptides on the cell membranes in the animal's body. Further, to the model mouse in which tumors were formed through the implantation of the HT1080 cells and the Hepa1-6 cell to the subcutaneous parts by injection, the anti-HA-tag peptide antibodies fluorescently labeled with Alexa 750 and the anti-c-Myc-tag peptide antibodies fluorescently labeled with Alexa 680 each were dosed at 15 □g/animal by intravenous injection to react with the HT 1080 cell tumor which expressed HA-tag peptides on the cell membranes or the Hepa1-6 cell tumor which expressed HA-tag peptides and c-Myc-tag peptides on the cell membranes.
7. After three days from the dosage of the fluorescently labeled antibodies, bright field images and fluorescence images of the fluorescence wavelength bands of Alexa680 and Alexa750 of the respective mice were captured with an in vivo bio-observation system OV110 (a system constructed by combining a stereoscopic microscope and a fluorescence image capturing device).

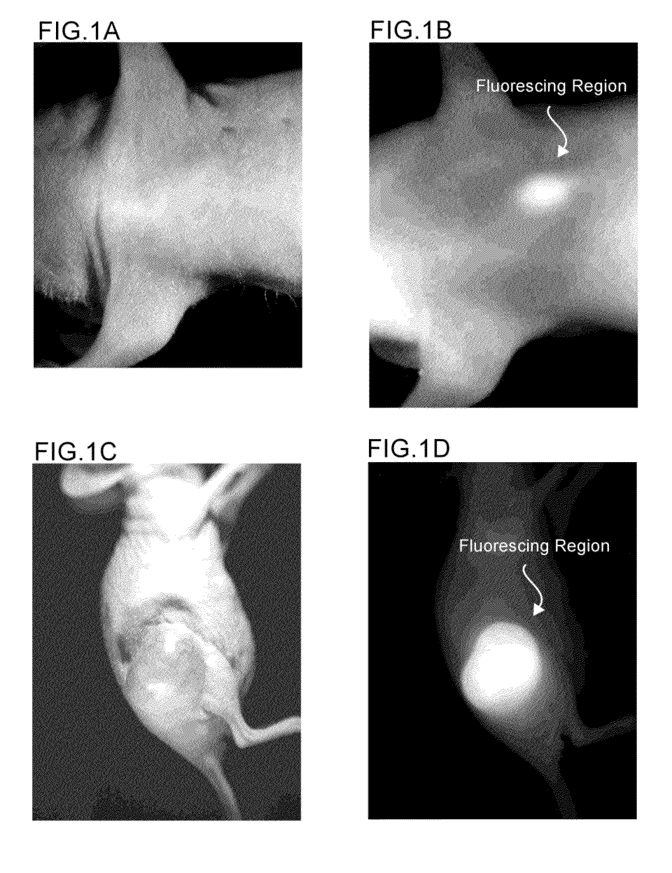

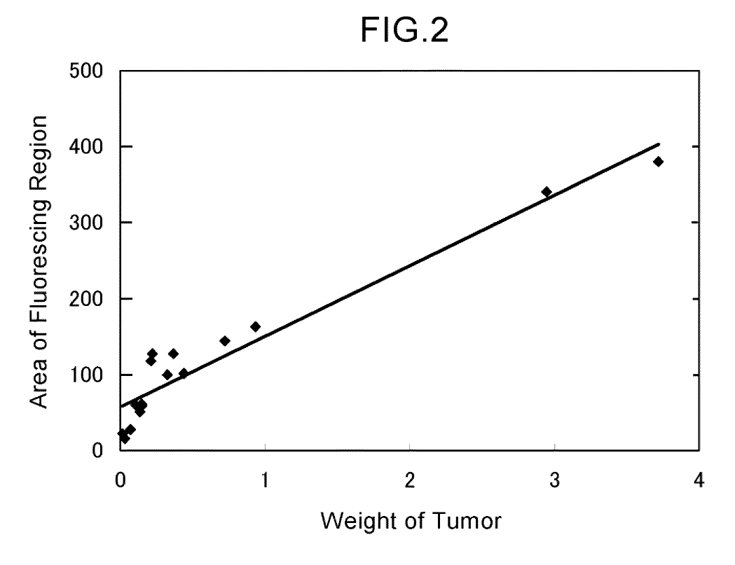

FIG. 1A-1D show bright field images and fluorescence images after 3 days (FIG. 1A, FIG. 1B) and after 14 days (FIG. 1C, FIG. 1D) from the implantation of the cells into model mice, into which the HT1080 cells, prepared to express HA-tag peptides on the cell membranes, had been implanted by subcutaneous injection and the anti-HA-tag peptide antibodies, fluorescently labeled with Alexa 750, were dosed according to the above-mentioned procedures. As apparent form the fluorescence images of FIG. 1B and FIG. 1D, it has been shown that the luminance became high on the sites where a tumor was considered to form (Fluorescence emitting region). Then, in accordance with the comparison of areas of the fluorescence existing regions (Fluorescence emitting region) in the fluorescence images (computed with software in the bio-observation system) and weights of the tumors extracted from the corresponding fluorescence existing regions in a plurality of animal individuals, the areas of the fluorescence existing regions increased with the weights of the tumors as shown in FIG. 2. This result suggests that it is possible to measure the size, position, etc. of implanted cells with an area of a fluorescing region, and indicates that the inventive method enables the evaluation of a tumor size.

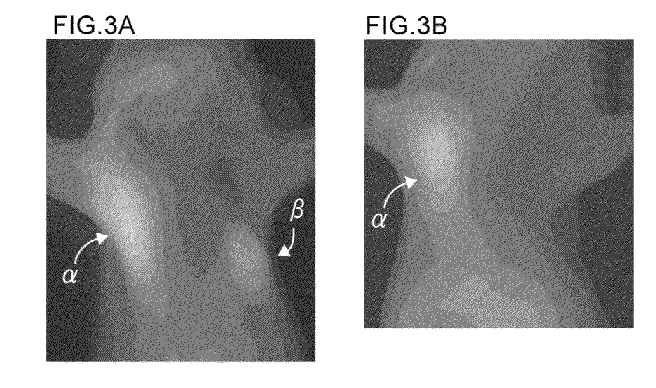

Further, FIG. 3A-FIG. 3B show fluorescence images of a model mouse, to which HT1080 cells prepared to express HA-tag peptides on the cell membranes and Hepa1-6 cells prepared to express both HA-tag peptides and c-Myc-tag peptides on the cell membranes had been implanted to subcutaneous parts by injection, respectively, and the anti-HA-tag peptide antibodies fluorescently labeled with Alexa 750 and the anti-c-Myc-tag peptide antibodies fluorescently labeled with Alexa 680 were dosed in accordance with the above-mentioned procedures. In these figures, FIG. 3A is a fluorescence image of the fluorescence wavelength band of Alexa 750, and FIG. 3B is a fluorescence image of the fluorescence wavelength band of Alexa 680. As apparent from these fluorescence images, in the fluorescence image of the fluorescence wavelength band of Alexa750, the fluorescence was emitted from two sites, while, in the fluorescence image in the fluorescence wavelength band of Alexa 680, the fluorescence was emitted from only one site corresponding to one of the sites emitting the fluorescence in the fluorescence image of the fluorescence wavelength band of Alexa 750. In the present sample, it is expected that the region occupied by HT 1080 cell tumor emits the fluorescence only of the fluorescence wavelength band of Alexa 750, while the region occupied by Hepa1-6 cell tumor emits the fluorescence of both the fluorescence wavelength band of Alexa 750 and the fluorescence wavelength band of Alexa 680, and therefore, from the results of FIG. 3A-FIG. 3B, it is estimated that the region □ fluorescing in both FIG. 3A and FIG. 3B is the region occupied by the Hepa1-6 cell tumor and the region □ fluorescing only in FIG. 3A is the region occupied by the HT 1080 cell tumor. Further, this result indicates that, according to the inventive method, it is possible to implant a plurality of kinds of cells into one animal, and to distinguish those implanted cells from one another in their observation.

Although the above explanations have been made with respect to the embodiments of the present invention, it should be apparent for one of ordinary skill in the art that modifications and variations can be possible and the present invention is not limited only to the embodiments illustrated above and can be applied to various cases without deviating from the scope of the present invention.

For instance, although, in the above-mentioned embodiment, a region occupied by implanted cells is detected non-invasively from the outside of an animal by fluorescence imaging, the imaging may be carried out while the neighborhood of implanted cells is exposed. In that case, it is advantageous in that the kind of an implanted cell can be specified only by determining its fluorescence wavelength. Further, according to the present invention, as a possible experimental manner, it is also possible to prepare a plurality of groups of tumor cells of a single species such that mutually different tags are made expressed in the respective groups; to implant the cell groups expressing mutually different tags into different sites, in different timings and/or by different methods; and to track and observe the respective implanted cell groups. The important point is that, according to the present invention, the range of freedom in the selection of kinds of marker for implanted cells becomes wider than ever, so that various experimental patterns which had been difficult to perform in the past become possible.

1. A method of in vivo detecting implanted cells implanted into a body of a small animal, comprising steps of:
preparing cells used as the implanted cells into which a gene expressing a tag of a part of a peptide or a protein on a cell membrane is introduced;
implanting the implanted cells to an arbitrary site of the small animal; and
detecting the tags in the small animal.

2. A method of claim 1, wherein a plurality of kinds of cells expressing mutually different tags are prepared as the implanted cells; the plurality of kinds of cells are implanted to a single small animal individual; and the mutually different tags each are detected in the small animal individual.

3. A method of claim 1, wherein, in the step of detecting the tags in the small animal, the tags are optically detected from an outside of the small animal.

4. A method of claim 1, wherein, in the step of detecting the tags in the small animal, an image of the small animal is acquired and a region occupied by the tags is detected in the image.

5. A method of claim 1, wherein, in the step of detecting the tags in the small animal, antibodies to the tags are dosed to the small animal and the tags are detected by detecting the antibodies binding to the tags.

6. A method of claim 1, wherein, in the step of detecting tags in the small animal, the tags are fluorescently labeled, and the tags are detected by detecting fluorescence from the tags.

7. A method of claim 6, wherein, in the step of detecting tags in the small animal, a fluorescence image of the small animal is acquired by carrying out a fluorescence observation of the small animal, and the tags are detected by detecting a region where fluorescence from the tags exists in the fluorescence image.

8. A method of claim 1, wherein, in the step of detecting tags in the small animal, fluorescently labeled antibodies to the tags are dosed to the small animal, and the tags are detected by detecting fluorescence from the antibodies binding to the tags.

9. A method of claim 8, wherein, in the step of detecting tags in the small animal, a fluorescence image of the small animal is acquired by carrying out a fluorescence observation of the small animal, and the tags are detected by detecting a region where fluorescence from the tags exits in the fluorescence image.

10. A method of claim 1, wherein, in the step of detecting tags in the small animal, a position or a spatial spread of the implanted cells is measured based on the detected region occupied by the tags.

11. A method of claim 1, wherein the tags are selected from a group consisting of a protein having an antigenicity, HA-tag peptide, Myctag peptide, His-tag peptide, T7-tag peptide, Flag-tag peptide, metal affinity-tag peptide, V5-tag peptide, VSV-G-tag peptide, S-tag peptide, Glu-Glu-tag peptide, and HSV-tag peptide.

12. A method of claim 1, wherein the implanted cells are tumor cells of a mammalian origin.

13. A method of in vivo detecting implanted cells implanted into a body of a small animal, comprising steps of:
preparing cells used as the implanted cells into which a gene expressing a tag of a part of a peptide or a protein on a cell membrane is introduced;
implanting the implanted cells to an arbitrary site of the small animal; and
dosing the small animal with fluorescently labeled antibodies to the tags; and
acquiring a fluorescence image of the small animal and detecting a region occupied by the implanted cells by detecting a region where fluorescence from the antibodies binding to the tags exists in the fluorescence image.

14. A method of claim 13, wherein a plurality of kinds of cells expressing mutually different tags are prepared as the implanted cells; the plurality of kinds of cells are implanted to a single small animal individual; antibodies to each of the mutually different tags are labeled with fluorescent substances having mutually different fluorescence wavelengths; and regions where fluorescence from the antibodies having the mutually different fluorescence wavelength and binding to the mutually different tags in the small animal individual exist are detected, respectively, whereby regions occupied by the implanted cells are detected for the respective kinds of the cells.

15. A method of claim 13, wherein the tags are selected from a group consisting of a protein having an antigenicity, HA-tag peptide, Myc-tag peptide, His-tag peptide, T7-tag peptide, Flag-tag peptide, metal affinity-tag peptide, V5-tag peptide, VSV-G-tag peptide, S-tag peptide, Glu-Glu-tag peptide, and HSV-tag peptide.

16. A method of claim 13, wherein the implanted cells are tumor cells of a mammalian origin.
